(19) United States
(12) Patent Application Publication
(10) Pub. No.: US 2023/0104299 A1
(43) Pub. Date: Apr. 6, 2023

(54) COMPUTATIONAL APPROACHES TO ASSESSING CENTRAL NERVOUS SYSTEM FUNCTIONALITY USING A DIGITAL TABLET AND STYLUS

(71) Applicant: Linus Health, Inc., Waltham, MA (US)

(72) Inventors: John Langton, Waltham, MA (US); David Bates, Waltham, MA (US); Sean Tobyne, Waltham, MA (US); Joyce Gomes-Osman, Waltham, MA (US); Alvaro Pascual-Leone, Waltham, MA (US); Ali Jannati, Waltham, MA (US); Sameer Dhamne, Waltham, MA (US)

(21) Appl. No.: 17/936,708

(22) Filed: Sep. 29, 2022

Related U.S. Application Data
(60) Provisional application No. 63/250,066, filed on Sep. 29, 2021.

(51) Int. Cl. A61B 5/00; A61B 5/22

(52) U.S. Cl. CPC A61B 5/4058; A61B 5/225; A61B 5/7475

(57) ABSTRACT

Computational approaches to assess CNS functionality using a digital tablet and stylus are provided.

### CROSS REFERENCE TO RELATED APPLICATION(S)

This application claims priority from U.S. Provisional Patent Application No. 63/250,066 filed on 29 Sep. 2021 entitled COMPUTATIONAL APPROACHES TO ASSESSING CNS FUNCTIONALITY USING A DIGITAL TABLET AND STYLUS, which is hereby incorporated by reference.

### BACKGROUND

Embodiments of the present disclosure relate to assessment of central nervous system (CNS) functionality and, more specifically, to computational approaches to assessing CNS functionality using a digital tablet and stylus.

Neurological diseases are among the most critical societal challenges of our time. As of 2011, nearly 100 million Americans had a neurological disorder. Neurological disorders are a source of significant disability and costs to individuals, families, and health care systems. In 2014, the annual economic burden associated with the nine most prevalent neurological disorders (Alzheimer's Disease [AD] and Other Dementias, Chronic Low Back Pain, Stroke, Traumatic Brain Injury, Epilepsy, Multiple Sclerosis, Traumatic Spinal Cord Injury, and Parkinson's Disease [PD]) was 789 billion dollars, in the US alone. Neurological disorders are even more prevalent in older age, and thus are expected to continue to exponentially increase at the current demographic growth patterns. Only in the next 10 years, older adults will grow another 17 million in the US, to reach a total of 73 million individuals. This phenomenon has broader global implications: by 2050 the worldwide older adult population will double from what it was in 2015, from 8.5% to 16.7% of the total population by 2050.

In the current reactive model of healthcare, access to clinical experts is limited, and often leading to delays in the diagnostic and treatment trajectory. Successful responses to the challenges posed by increased prevalence in neurological diseases will thus require a shift toward a pre-emptive model, characterized by early detection and timely deployment of targeted, personalized interventions that can be scalable to meet these growing demands. For this reason, technology screening and assessment methods are appealing.

Handwriting and drawing are complex activities that require specific contributions of distinct brain networks, combining motor, cognitive, perceptual and contextual information that are necessary to reach the desired goals. Clinical instruments for screening many neurological disorders include handwriting as part of their assessments, but typically the final performance is the critical aspect that is incorporated into the score. In this context, a loss of fine motor function while drawing is known to be associated with dementia (in the early stages of Lewy Body Dementia, and in the later stages of AD), and a reduction in the size of handwriting (or micrographia) is known to be associated with PD.

In addition to these more global insights, the application of digital assessments and machine learning algorithms enable the quantification of more specific metrics, such as the pressure exerted on the pen, velocity, acceleration, pauses, thereby deconstructing the sequences of behaviors employed during the performance of each handwriting or drawing task. Emerging evidence highlights the value of this approach to gain greater insights into more subtle motor abnormalities that are below the threshold of clinical detection. For instance, handwriting analysis revealed significant differences in automation, relative velocity, and velocity variation while drawing concentric circles between healthy individuals and those with mild cognitive impairment and AD. In addition, stroke length, width, and height, mean pressure, mean time per stroke and mean velocity were all features that significantly distinguished healthy controls from individuals with PD.

Additional information about drawing tasks for assessment of CNS functionality, including clock drawing tasks, is provided in U.S. Pub. No. 2021/0295969, which is hereby incorporated by reference in its entirety.

### BRIEF SUMMARY

A computer-implemented method of predicting hand strength of a participant in accordance with one or more embodiments comprises: (a) receiving input data captured from performance of a task by the participant, said task comprising generating a drawing of an item on a computer display using a stylus, the input data including: (i) drawing data comprising timestamped X and Y coordinates of points on drawing on the computer display collected at a given rate as the drawing is generated, and (ii) stylus data including tip pressure, altitude, and azimuth of the stylus associated with each of the points; (b) processing the input data to generate derived metrics; and (c) providing the derived metrics to a pre-trained machine learning model to estimate the hand strength of the participant.

In accordance with one or more further embodiments, a non-transitory computer-readable medium storing instructions that, when executed by one or more computing devices, cause the one or more computing devices to perform a method of predicting hand strength of a participant. The method comprises receiving input data captured from performance of a task by the participant. The task comprises generating a drawing of an item on a computer display using a stylus, the input data including: (i) drawing data comprising timestamped X and Y coordinates of points on drawing on the computer display collected at a given rate as the drawing is generated, and (ii) stylus data including tip pressure, altitude, and azimuth of the stylus associated with each of the points. The input data is processed to generate derived metrics. The derived metrics are provided to a pre-trained machine learning model to estimate the hand strength of the participant.

In accordance with one or more further embodiments, a system is disclosed for predicting hand strength of a participant. The system includes a data storage device that stores instructions for predicting the hand strength of the participant. The system also includes a processor configured to execute the instructions to perform a method including (a) receiving input data captured from performance of a task by the participant, said task comprising generating a drawing of an item on a computer display using a stylus, the input data including: (i) drawing data comprising timestamped X and Y coordinates of points on drawing on the computer display collected at a given rate as the drawing is generated, and (ii) stylus data including tip pressure, altitude, and azimuth of the stylus associated with each of the points; (b) processing the input data to generate derived metrics; and (c) providing the derived metrics to a pre-trained machine learning model to estimate the hand strength of the participant.

### BRIEF DESCRIPTION OF THE SEVERAL VIEWS OF THE DRAWINGS

FIG. 1 illustrates an exemplary system architecture of a system for estimating hand strength of a participant according to embodiments of the present disclosure.

FIG. 2 illustrates an exemplary process for estimating hand strength of a participant according to embodiments of the present disclosure.

FIG. 3 depicts a computing node according to embodiments of the present disclosure.

### DETAILED DESCRIPTION

Various embodiments disclosed herein generally relate to methods for computational analysis of brain function by analysis of handwriting behaviors using a scientifically- and medically-informed algorithm(s) that takes into account inputs derived from sensors embedded in commercially available digital tablets and their accompanying stylus. The advantage of this method is to analyze additional aspects of brain function, passively, while the user is undertaking prescribed, tablet-based assessments. Automated handwriting analysis provides a means for extracting clinically relevant features and outcomes in addition to the core metrics for a given assessment (e.g., time to complete or accuracy) without placing additional burden on the participant.

In particular, various embodiments disclosed herein relate to methods and systems for estimating grip strength and pinch strength, which are key components of the ability to perform tasks requiring fine motor skills. These skills can degrade with age, and could be an early indicator of frailty, which is associated with declining long term outcomes for older adults at risk for dementia. According to various embodiments, grip and pinch strength are predicted from drawing tasks performed with a tablet and paired stylus by analyzing a participant's drawing, the process of creating that drawing (e.g., speed/velocity, size, component placements), and use of the drawing stylus (e.g., stylus tip force, altitude, and azimuth).

The system works by tracking metrics native to the tablet and its associated stylus (e.g., altitude, azimuth, pressure) while the participant performs one of a set of stylus drawing tasks (e.g., a clock drawing test or other tests described in U.S. Pub. No. 2021/0295969, which is hereby incorporated by reference in its entirety). Each stylus drawing task includes associated core metrics (e.g., number of strokes, stylus speed, drawing size) as appropriate for the given task. Stylus metrics are collected as additional sources of participant information seamlessly while the participant focuses on the given task. The core metrics associated with the task may be used in other algorithms not described here. Once the assessment is complete, it is packaged and transferred to the cloud data lake. From there, assessment specific core metrics and stylus metrics are extracted, processed, and featurized. Stylus metrics are then passed into a pre-trained machine learning model to estimate hand strength from multivariate stylus features, before estimating a frailty score as a final model output.

In various embodiments, metrics include:

### P=Pressure

### Z=Azimuth

### A=Altitude

X=X-coordinate on tablet
Y=Y-coordinate on tablet
V=velocity of stylus
d=distance that stylus writing tip traveled across tablet screen
D=distance non-writing end of stylus traveled while writing on the tablet screen

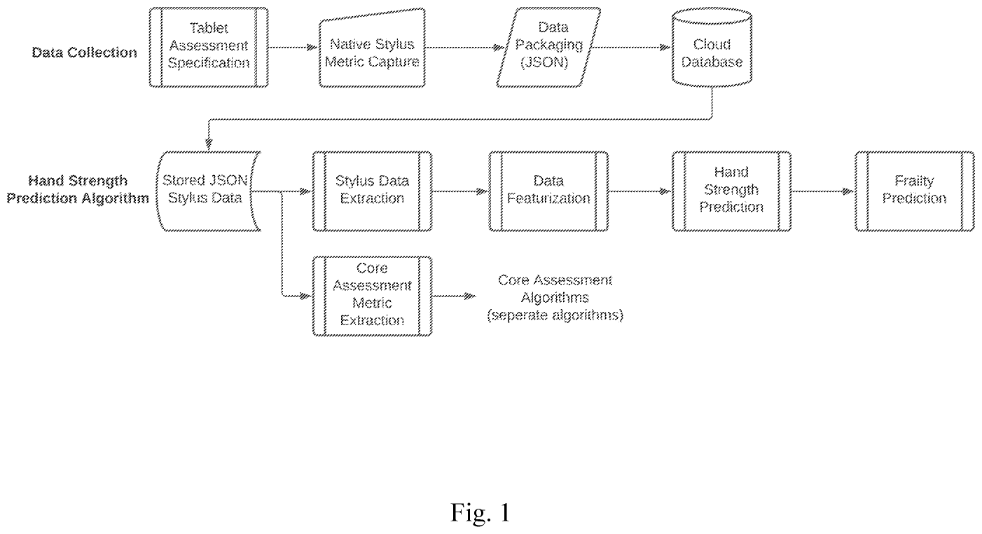

FIG. 1 illustrates an exemplary system architecture of a system for estimating hand strength of a participant according to embodiments of the present disclosure. Data capture components of the system include a tablet 102 and a stylus 104 (which can, e.g., be paired to the tablet 102 through, e.g., a Bluetooth connection). The tablet 102 runs a clock drawing test application (e.g., the clock drawing test described in U.S. Pub. No. 2021/0295969). In one or more embodiments, the application is a standard Linus Health DCTclock assessment test capable of acquiring DCTclock assessments. The stylus 104 is capable of recording stylus tip pressure/force, altitude, and azimuth data.

Raw data from the tablet 102 is uploaded from the tablet 102 to a DCTclock module 106, which includes a DCTclock data processing engine 108, a database for storing participant demographic data, and a system for queuing and tracking data processing.

A hand strength module 110, includes hand strength data featurization and modeling components, including a hand strength prediction engine 112, a database for retrieving participant information and storing model outputs, and a model repository 114. In one or more embodiments, the model architecture utilizes a standard gradient boosting ensemble method. Models are stored within a model registry 114 and imported into the hand strength prediction engine 112.

A data output module 116 includes data export and downstream processing components, including a system for exporting data to a data lake 118. A recommendation engine 120 suggests applicable recommendations from model outputs. A report engine 122 generates reports for downstream functions 124, e.g., reports to medical professionals.

In one or more embodiments, the raw data and derived metrics are processed by a cloud-native system implemented, e.g., in AWS, immediately upon upload from the tablet application.

Following processing, raw data, derived metrics, and model outputs are entered in the cloud data lake 118 for archiving and later analysis. In parallel, the model output can be used by Linus Health's reporting module to present the outcomes and recommendations to medical professionals in near-real time (i.e., within seconds).

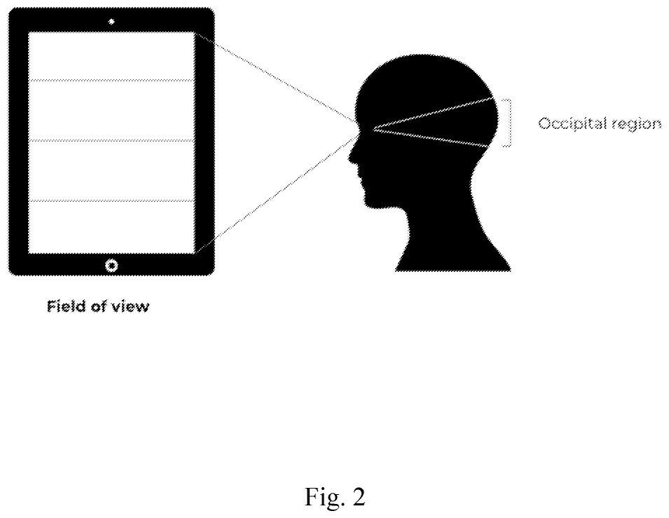

FIG. 2 illustrates an exemplary process 200 for estimating hand strength of a participant according to embodiments of the present disclosure. At step 210, input data is generated from performance of a clock drawing task by the participant. The clock drawing task is performed on a computer display of a tablet using a stylus paired to the tablet. The input data includes drawing data comprising timestamped X and Y coordinates of points on drawing on the computer display collected at a given rate (e.g., 120-240 Hz.) as the drawing is generated. These coordinates are used to reconstruct the participant's drawing. The input also includes stylus data including tip pressure, altitude, and azimuth of the stylus associated with each of the points. At step 220, the input data is processed to generate derived metrics. At step 230, the derived metrics are provided to a pre-trained machine learning model to estimate the hand strength of the participant. At step 240, the hand strength data is output, e.g., to medical professionals in near-real time.

In one or more embodiments, raw data are extracted from a JSON body and processed into derived metrics using custom Python software. First, the raw coordinate data is processed and classified with computer vision algorithms to identify the stroke or strokes that make up the clock face. Data are combined as necessary to derive a single clock face raw dataset. Average stylus pressure is calculated across all time points attributed to the clock face. Next the clock face stroke data is divided into four equal quarters. If an odd number of time points exist, the odd time point is attributed to the first quarter of the stroke. The indices from the division of the stroke into quarters are then used to parse the stylus pressure and average over the quarters, producing an average stylus pressure for each of the four quarters. The difference in pressure between quarters is then calculated. Determining pressure differences is important because participants experiencing issues with fine motor control, strength, coordination, or frailty will demonstrate greater deviance between the start of the drawing stroke and later portions of the drawing stroke. After all derived metrics are calculated, they are normalized to the group mean with unit variance by calculating z-scores for the training data set. The mean and standard deviation calculated for the training data set are applied to the testing dataset during model evaluation.

Several machine learning models can be used herein for estimating continuous variables from multivariate feature sets. In one or more embodiments, random forest regression and gradient boosting ensemble model types may be used. In one or more embodiments, the model types are ‘off-the-shelf’ capabilities of the scikit-learn Python package custom tuned to optimize performance for the application and available dataset.

In one or more embodiments, the system output uses a 0-200 lbs. numeric scale for estimating grip strength and a 0-45 lbs. numeric scale for estimating pinch strength.

In one or more embodiments, the parameters of a gradient boosting model for predicting hand strength are as follows:
learning rate=0.1
maximum features=3
number of estimators=3
subsample=0.4
maximum depth=5

### Exemplary Model Development and Data Analysis

A data sample was collected from 21 healthy adult participants (6 females) to support the development of a proof-of-concept system. Isometric grip strength was recorded as an integer ranging from 0-200 lbs. using a hand-held hydraulic dynamometer and pinch strength was recorded on a scale of 0-45 lbs. using a hydraulic pinch gauge to estimate the maximum force of the grip or pinch, respectively. Three sets of three trials each were conducted for each participant in the test procedure. These trials were averaged to produce a continuous float variable of maximum grip or pinch strength. In total, the process produced 64 grip and pinch stretch samples from the 21 participants. In addition to grip and pinch strength measurements, participants also performed the DCTclock assessment three times before the strength measurements. Data was processed using the procedure outlined above. Raw DCTclock data was extracted from the JSON body and processed into derived metrics using custom Python software. First, raw coordinate data was processed and classified with computer vision algorithms to identify the stroke or strokes that make up the clock face. Data were combined as necessary to derive a single clock face raw dataset. Average stylus pressure was calculated across all time points attributed to the clock face. Next the clock face stroke data was divided into four equal quarters. If an odd number of time points exist, the odd time point is attributed to the first quarter of the stroke. The indices from the division of the stroke into quarters were then used to parse the stylus pressure and average over the quarters, producing an average stylus pressure for each of the four quarters. The difference between quarters was then calculated. After all derived metrics were calculated, they were normalized to the group mean with unit variance by calculating z-scores for the training data set. The mean and standard deviation calculated for the training data set were applied to the testing dataset during model evaluation. Following data normalization, featurized stylus pressure data were combined with a binarized variable representing gender.

### Results Summary

Group statistics, prior to normalization, are described in Table 1 below.

| | q1_mean_pres- | q2_mean_pres- | q3_mean_pres- | q4_mean_pres- | | | | |
|---|---|---|---|---|---|---|---|---|
| | sure | sure | sure | sure | avg_grip | avg_pinch | q4q1_difference | q4q2_difference |
| count | 58.000000 | 58.000000 | 58.000000 | 58.000000 | 58.000000 | 58.000000 | 58.000000 | 58.000000 |
| mean | 0.979225 | 1.366209 | 1.606220 | 1.678843 | 92.106322 | 19.414368 | 0.699519 | 0.322634 |
| std | 0.813320 | 0.840999 | 0.871990 | 0.911139 | 28.822442 | 4.755121 | 0.497861 | 0.432743 |

Table 1 shows group statistics for grip and pinch strength measurements, as well as model features.

The total dataset was split into a training and testing sample to diminish the effects of overfitting. Five of the total 21 subjects (24%) were randomly assigned to the testing sample. Features distributions were not significantly different between training and testing samples (all p-values >0.21).

Several model types were evaluated. Gradient boosting ensemble methods were superior to all tested models. A grid search paradigm with five-fold cross validation was used to tune the model over the following parameter distributions:
Maximum depth: [1, 3, 5, 7, 9, 11, 13, 15]
Number of estimators: [1, 3, 5, 7, 10, 20, 50]
Subsampling: [0.1, 0.2, 0.3, 0.4, 0.5, 0.6, 0.7, 0.8, 0.9]
   Best parameters:
Maximum depth: 5
Number of estimators: 3
Subsampling: 0.4
   The best performing model produced a mean squared error of 5.51.

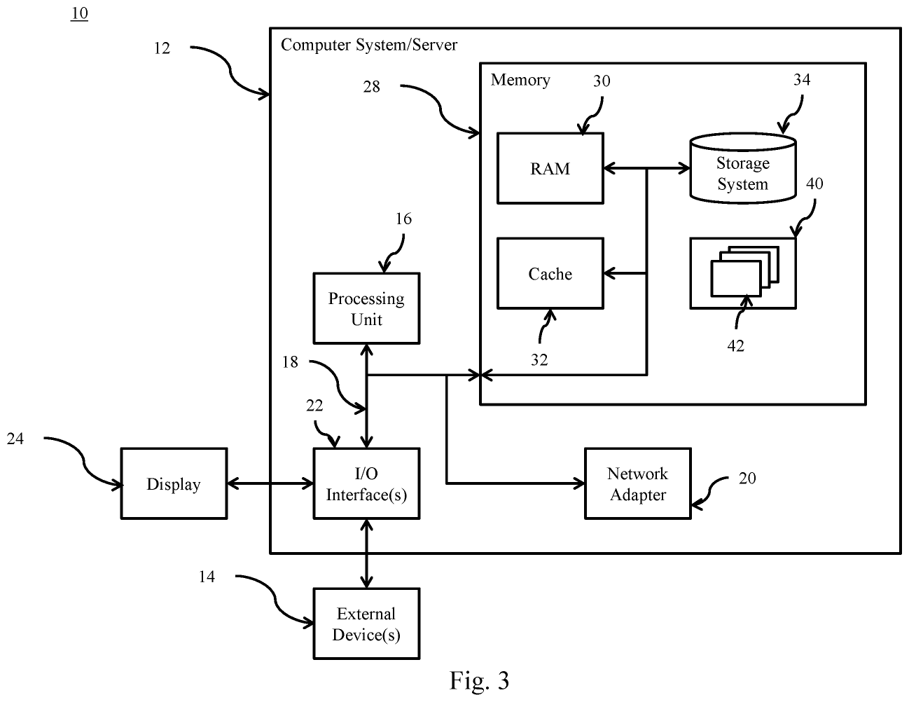

Referring now to FIG. 3, a schematic of an example of a computing node is shown. Computing node 10 is only one example of a suitable computing node and is not intended to suggest any limitation as to the scope of use or functionality of embodiments described herein. Regardless, computing node 10 is capable of being implemented and/or performing any of the functionality set forth hereinabove.

In computing node 10 there is a computer system/server 12, which is operational with numerous other general purpose or special purpose computing system environments or configurations. Examples of well-known computing systems, environments, and/or configurations that may be suitable for use with computer system/server 12 include, but are not limited to, personal computer systems, server computer systems, thin clients, thick clients, handheld or laptop devices, multiprocessor systems, microprocessor-based systems, set top boxes, programmable consumer electronics, network PCs, minicomputer systems, mainframe computer systems, and distributed cloud computing environments that include any of the above systems or devices, and the like.

Computer system/server 12 may be described in the general context of computer system-executable instructions, such as program modules, being executed by a computer system. Generally, program modules may include routines, programs, objects, components, logic, data structures, and so on that perform particular tasks or implement particular abstract data types. Computer system/server 12 may be practiced in a distributed cloud computing environments where tasks are performed by remote processing devices that are linked through a communications network. In a distributed cloud computing environment, program modules may be located in both local and remote computer system storage media including memory storage devices.

As shown in FIG. 3, computer system/server 12 in computing node 10 is shown in the form of a general-purpose computing device. The components of a computer system/server 12 may include, but are not limited to, one or more processors or processing units 16, a system memory 28, and a bus 18 that couples various system components including system memory 28 to processor 16.

Bus 18 represents one or more of any of several types of bus structures, including a memory bus or memory controller, a peripheral bus, an accelerated graphics port, and a processor or local bus using any of a variety of bus architectures. By way of example, and not limitation, such architectures include Industry Standard Architecture (ISA) bus, Micro Channel Architecture (MCA) bus, Enhanced ISA (EISA) bus, Video Electronics Standards Association (VESA) local bus, Peripheral Component Interconnect (PCI) bus, Peripheral Component Interconnect Express (PCIe), and Advanced Microcontroller Bus Architecture (AMBA).

Computer system/server 12 typically includes a variety of computer system readable media. Such media may be any available media that is accessible by computer system/server 12, and it includes both volatile and non-volatile media, removable and non-removable media.

System memory 28 can include computer system readable media in the form of volatile memory, such as random access memory (RAM) 30 and/or cache memory 32. Computer system/server 12 may further include other removable/non-removable, volatile/non-volatile computer system storage media. By way of example only, storage system 34 can be provided for reading from and writing to a non-removable, non-volatile magnetic media (not shown and typically called a “hard drive”). Although not shown, a magnetic disk drive for reading from and writing to a removable, non-volatile magnetic disk (e.g., a “floppy disk”), and an optical disk drive for reading from or writing to a removable, non-volatile optical disk such as a CD-ROM, DVD-ROM or other optical media can be provided. In such instances, each can be connected to bus 18 by one or more data media interfaces. As will be further depicted and described below, memory 28 may include at least one program product having a set (e.g., at least one) of program modules that are configured to carry out the functions of embodiments of the disclosure.

Program/utility 40, having a set (at least one) of program modules 42, may be stored in memory 28 by way of example, and not limitation, as well as an operating system, one or more application programs, other program modules, and program data. Each of the operating system, one or more application programs, other program modules, and program data or some combination thereof, may include an implementation of a networking environment. Program modules 42 generally carry out the functions and/or methodologies of embodiments as described herein.

Computer system/server 12 may also communicate with one or more external devices 14 such as a keyboard, a pointing device, a display 24, etc.; one or more devices that enable a user to interact with computer system/server 12; and/or any devices (e.g., network card, modem, etc.) that enable computer system/server 12 to communicate with one or more other computing devices. Such communication can occur via Input/Output (I/O) interfaces 22. Still yet, computer system/server 12 can communicate with one or more networks such as a local area network (LAN), a general wide area network (WAN), and/or a public network (e.g., the Internet) via network adapter 20. As depicted, network adapter 20 communicates with the other components of computer system/server 12 via bus 18. It should be understood that although not shown, other hardware and/or software components could be used in conjunction with computer system/server 12. Examples, include, but are not limited to: microcode, device drivers, redundant processing units, external disk drive arrays, RAID systems, tape drives, and data archival storage systems, etc.

The present disclosure may be embodied as a system, a method, and/or a computer program product. The computer program product may include a computer readable storage medium (or media) having computer readable program instructions thereon for causing a processor to carry out aspects of the present disclosure.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present disclosure may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Smalltalk, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present disclosure.

Aspects of the present disclosure are described herein with reference to flowchart illustrations and/or block diagrams of methods, apparatus (systems), and computer program products according to embodiments of the disclosure. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present disclosure. In this regard, each block in the flowchart or block diagrams may represent a module, segment, or portion of instructions, which comprises one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

The descriptions of the various embodiments of the present disclosure have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

What is claimed is:

1. A computer-implemented method of predicting hand strength of a participant, comprising:
(a) receiving input data captured from performance of a task by the participant, said task comprising generating a drawing of an item on a computer display using a stylus, the input data including: (i) drawing data comprising timestamped X and Y coordinates of points on drawing on the computer display collected at a given rate as the drawing is generated, and (ii) stylus data including tip pressure, altitude, and azimuth of the stylus associated with each of the points;
(b) processing the input data to generate derived metrics; and
(c) providing the derived metrics to a pre-trained machine learning model to estimate the hand strength of the participant.

2. The method of claim 1, wherein the task is a clock drawing test.

3. The method of claim 2, wherein the clock drawing test includes drawing one or more of hour labels, an hour hand, a minute hand, a second hand, a clock face outline, and a clock face center point.

4. The method of claim 2, wherein the derived metrics include average pressure for strokes in each quarter of a clock face drawn in the clock drawing test, and differences in pressure between at least two of the quarters.

5. The method of claim 1, wherein the hand strength comprises grip or pinch strength.

6. The method of claim 1, wherein the hand strength is indicative of motor skills or cognitive skills of the participant.

7. The method of claim 1, wherein the hand strength is indicative of frailty of the participant.

8. The method of claim 1, wherein processing the input data to generate derived metrics includes processing and classifying the drawing data using computer vision algorithms to identify one or more strokes that make up the drawing.

9. The method of claim 8, wherein the derived metrics include at least one of speed of the one or more strokes, size of the one or more strokes, and drawing component placements.

10. The method of claim 1, further comprising outputting the estimated hand strength of the participant to medical professionals in near-real time.

11. A non-transitory computer-readable medium storing instructions that, when executed by one or more computing devices, cause the one or more computing devices to perform a method of predicting hand strength of a participant, the method comprising:
receiving input data captured from performance of a task by the participant, said task comprising generating a drawing of an item on a computer display using a stylus, the input data including: (i) drawing data comprising timestamped X and Y coordinates of points on drawing on the computer display collected at a given rate as the drawing is generated, and (ii) stylus data including tip pressure, altitude, and azimuth of the stylus associated with each of the points;
processing the input data to generate derived metrics; and
providing the derived metrics to a pre-trained machine learning model to estimate the hand strength of the participant.

12. The non-transitory computer-readable medium of claim 11, wherein the task is a clock drawing test.

13. The non-transitory computer-readable medium of claim 12, wherein the clock drawing test include drawing one or more of hour labels, an hour hand, a minute hand, a second hand, a clock face outline, and a clock face center point.

14. The non-transitory computer-readable medium of claim 12, wherein the derived metrics include average pressure for strokes in each quarter of a clock face drawn in the clock drawing test, and differences in pressure between at least two of the quarters.

15. The non-transitory computer-readable medium of claim 11, wherein the hand strength comprises grip or pinch strength.

16. The non-transitory computer-readable medium of claim 11, wherein the hand strength is indicative of motor skills or cognitive skills of the participant.

17. The non-transitory computer-readable medium of claim 11, wherein the hand strength is indicative of frailty of the participant.

18. The non-transitory computer-readable medium of claim 12, wherein processing the input data to generate derived metrics includes processing and classifying the drawing data using computer vision algorithms to identify one or more strokes that make up the drawing.

19. The non-transitory computer-readable medium of claim 18, wherein the derived metrics include at least one of speed of the one or more strokes, size of the one or more strokes, and drawing component placements.

20. A system for predicting hand strength of a participant, the system including:
a data storage device that stores instructions for predicting the hand strength of the participant; and
a processor configured to execute the instructions to perform a method including:
receiving input data captured from performance of a task by the participant, said task comprising generating a drawing of an item on a computer display using a stylus, the input data including: (i) drawing data comprising timestamped X and Y coordinates of points on the drawing on the computer display collected at a given rate as the drawing is generated, and (ii) stylus data including tip pressure, altitude, and azimuth of the stylus associated with each of the points;
processing the input data to generate derived metrics; and
providing the derived metrics to a pre-trained machine learning model to estimate the hand strength of the participant.

21. A computer-implemented method of assessing frailty of a participant, comprising:
(a) receiving input data captured from performance of a task by the participant, said task comprising generating a drawing of an item on a computer display using a stylus, the input data including: (i) drawing data comprising time-stamped X and Y coordinates of points on the drawing on the computer display collected at a given rate as the drawing is generated, and (ii) stylus data including tip pressure, altitude, and azimuth of the stylus associated with each of the points;
(b) processing the input data to generate derived metrics; and
(c) providing the derived metrics to a pre-trained machine learning model to estimate the hand strength of the participant to predict the frailty of the participant.
